(19) United States
(12) Patent Application Publication
(10) Pub. No.: US 2026/0038694 A1
(43) Pub. Date: Feb. 5, 2026

(54) SYSTEM AND METHOD FOR DETERMINING COGNITIVE HEALTH STATE BASED ON FUNCTIONAL NEAR-INFRARED SPECTROSCOPY

(71) Applicant: The Chinese University of Hong Kong, Shatin (HK)

(72) Inventors: Sui-yin Agnes Chan, Tai Wai (CN); Tsz-lok Lee, New Territories (CN)

(21) Appl. No.: 19/284,038

(22) Filed: Jul. 29, 2025

Related U.S. Application Data
(60) Provisional application No. 63/677,560, filed on Jul. 31, 2024.

(51) Int. Cl. G16H 50/30; A61B 5/00; A61B 5/1455

(52) U.S. Cl. CPC G16H 50/30; A61B 5/14553; A61B 5/4088

(57) ABSTRACT

A computer-implemented method for determining cognitive health state of a subject. The method includes receiving fNIRS data of the subject. The fNIRS data is acquired using a system operable to perform fNIRS, and the fNIRS data contains information associated with cognitive task related cerebral hemodynamics of the subject. The method further includes processing the fNIRS data to obtain cognitive task related cerebral hemodynamics data, processing the cognitive task related cerebral hemodynamics data using at least a model, and determining, based at least in part on the processing of the cognitive task related cerebral hemodynamics data, the cognitive health state of the subject.

### TECHNICAL FIELD

This invention relates to system and method for determining cognitive health state of a subject based on functional near-infrared spectroscopy (fNIRS).

### BACKGROUND

Dementia refers to an individual's decline in cognitive function to an extent that affects daily life and activities. Currently, dementia affects tens of millions of people worldwide, and it poses significant burden to individuals who suffer from it, their caregivers, and the society in general.

Dementia is generally progressive and may broadly include four stages: normal aging (normal cognition (NC)), subjective memory complaint (SMC), mild cognitive impairment (MCI), and dementia. Individuals with SMC usually have self-perceived or subjective cognitive decline. When compared with individuals without SMC, individuals with SMC have a higher risk of developing dementia. Further, when compared with individuals under normal aging, individuals with SMC may exhibit an increased risk of abnormalities in dementia-related biomarkers, regional brain hypometabolism, and/or atrophy in the medial temporal lobe. On the other hand, individuals with MCI exhibit lower performance on neuropsychological assessments but can maintain independent living abilities. MCI can be further categorized into amnestic MCI (aMCI), characterized by memory impairment, and non-amnestic MCI (naMCI), characterized by impairments in cognitive domains other than memory. aMCI may be more predictive of Alzheimer's disease (AD) whereas naMCI may be more predictive of other dementia subtypes.

Recent advancements in pharmacological interventions for early-stage dementia have highlighted the importance of identifying early signs of dementia before the final dementia stage is reached.

Conventionally, the diagnosis of MCI and dementia is based heavily on clinical diagnosis using standardized neuropsychological tests and clinical interviews. A problem associated with this approach is that it can be labor intensive.

To address this problem, more recently, the detection of the preclinical stage of dementia involves identifying abnormalities associated with related biomarkers. To date, the core biomarkers for dementia predominantly depend on positron emission tomography (PET) and the analysis of cerebrospinal fluid (CSF) and plasma samples. These indicators encompass alterations in A (amyloid beta) and T (tau) that can be identified through PET scans, which are useful for determining the transition/stage associated with dementia. However, a problem associated with this approach is that it is invasive (e.g., to obtain the samples).

### SUMMARY

In a first aspect, there is provided a computer-implemented method determining cognitive health state of a subject. The computer-implemented method comprises receiving functional near-infrared spectroscopy (fNIRS) data of a subject. The fNIRS data is acquired from the subject using a system operable to perform fNIRS. The system includes multiple optodes operable as light sources and light detectors (or simply, sources and detectors). The fNIRS data contains information associated with cognitive task related cerebral hemodynamics of the subject (and optionally further information). The computer-implemented method further comprises processing the fNIRS data to obtain cognitive task related cerebral hemodynamics data, processing the cognitive task related cerebral hemodynamics data using at least a model, and determining, based at least in part on the processing of the cognitive task related cerebral hemodynamics data, the cognitive health state of the subject.

The fNIRS data is obtained from the subject using the system when the subject is performing a cognitive task. In some embodiments, the cognitive task related cerebral hemodynamics data is associated with hemodynamics of a brain region of the subject. The brain region includes the prefrontal cortex region (and optionally other region(s)).

In some embodiments, the cognitive task related cerebral hemodynamics data comprises cognitive task related oxyhemoglobin (HbO) data and/or cognitive task related deoxyhemoglobin (HbR) data.

In some embodiments, the cognitive task comprises a visual memory span task including a plurality of trials, each trial comprising a control task and a visual memory span cognitive task.

In some embodiments, the processing of the fNIRS data comprises: (i) performing a data extraction operation on the fNIRS data to obtain light intensity data; (ii) performing a data conversion operation on the light intensity data to obtain optical density data; (iii) performing a transformation operation on the optical density data to obtain HbO data containing information associated with relative HbO concentration changes and/or HbR data containing information associated with relative HbR concentration changes; (iv) performing a correlation-based adjustment operation and a baseline correction operation on the HbO data and/or the HbR data, to obtain modified HbO data and/or modified HbR data; and (v) performing an averaging operation on the modified HbO data and/or the modified HbR data to obtain the cognitive task related cerebral hemodynamics data.

In some embodiments, performing the data extraction operation comprises: extracting, from the fNIRS data, parameters associated with source-detector geometry of the system, information associated with stimulus onsets of the cognitive task, information associated source-detector channels of the system, and the light intensity data comprising data time points and raw light intensity measurements of each of the channels of the system. In some embodiments, performing the data extraction operation further comprises further extracting, from the fNIRS data, auxiliary signal.

In some embodiments, the data conversion operation is performed based at least in part on:

$$O D = - log \frac{\vert d \vert}{\vert {mean}_{d} \vert}$$

where OD is associated with the optical density data of a channel, d is associated with the light intensity data of the channel, and mean_{d} is associated with mean light intensity of the channel.

In some embodiments, performing the transformation operation comprises: processing the optical density data based at least in part on modified Beer-Lambert law. In some embodiments, performing the transformation operation comprises: processing the optical density data based at least in part on absorption coefficient values and distance factors.

In some embodiments, performing the correlation-based adjustment operation and the baseline correction operation comprises performing the correlation-based adjustment operation prior to performing the baseline correction operation. In some embodiments, performing the correlation-based adjustment operation and the baseline correction operation comprises performing the correlation-based adjustment operation after performing the baseline correction operation.

In some embodiments, performing the correlation-based adjustment operation and the baseline correction operation comprises: performing the correlation-based adjustment operation on the HbO data and/or the HbR data to obtain correlation-adjusted HbO data and/or correlation-adjusted HbR data; and performing the baseline correction operation on the correlation-adjusted HbO data and/or the correlation-adjusted HbR data to obtain the modified HbO data and/or the modified HbR data.

In some embodiments, performing the correlation-based adjustment operation comprises: processing the HbO data and/or the HbR data based at least in part on a correlation function arranged to effectuate negative correlation between concentration changes of HbO and concentration changes of HbR.

In some embodiments, performing the baseline correction operation comprises: processing the HbO data and/or the HbR data for each trial based at least in part on data obtained during the control task.

In some embodiments, performing the averaging operation comprises: averaging the modified HbO data and/or the modified HbR data for each trial based at least in part on data obtained during the visual memory span cognitive task to obtain averaged HbO data and/or averaged HbR data for each trial; averaging the averaged HbO data and/or averaged HbR data for each trial across trials with the same condition, to obtain cognitive task based HbO data and/or cognitive task based HbR data; and averaging the cognitive task based HbO data and/or cognitive task based HbR data to across all channels that have not been pruned.

In some embodiments, the processing of the fNIRS data further comprises: prior to (ii), performing an intensity correction operation to remove all negative light intensity values from the light intensity data. In some embodiments, performing the intensity correction operation comprises: replacing each negative light intensity value with a respective distance value from 1.0 to a next integer double-precision number. In some embodiments, performing the intensity correction operation comprises: for each negative light intensity value, applying one or more increment signals to all negative light intensity values in the light intensity data. The intensity correction operation may not be performed if there are no negative light intensity values in the light intensity data.

In some embodiments, the processing of the fNIRS data further comprises: performing a channel pruning operation to prune one or more of the channels based at least in part on one or more criteria. In some embodiments, the channel pruning operation is performed prior to (ii). In some embodiments, for each channel, the one or more criteria are associated with light intensity values obtained from the channel and one or more thresholds. In some embodiments, for each channel, the one or more criteria are associated with: mean light intensity value of the light intensity values obtained from the channel, standard deviation of light intensity values obtained from the channel, one or more light intensity value thresholds, and a signal-to-noise ratio threshold. The channel pruning operation may not be performed if no channel pruning is required (as determined based at least in part on the one or more criteria).

In some embodiments, for each channel, the one or more criteria comprises at least one of: Mean_{d}>Rangeᵤₚₚₑᵣ, Mean_{d}<Range_{lower}, and

$$\frac{{Mean}_{d}}{S {D}_{d}} < S N {R}_{thresh} ,$$

where Mean_{d} represents mean light intensity value associated with the channel, SD_{d} represents standard deviation of light intensity values associated with the channel, Rangeᵤₚₚₑᵣ represents an upper light intensity value threshold, Range_{lower} represents a lower light intensity value threshold, and SNRₜₕᵣₑₛₕ represents the signal-to-noise ratio threshold, and the channel is pruned if any of the one or more criteria is met.

In some embodiments, the processing of the fNIRS data further comprises: prior to (iii), performing a filtering operation to at least partly remove noise from the optical density data. In one embodiments, performing the filtering operation comprises: processing the optical density data using a bandpass filter or a low pass filter. In one example, the filter comprises an n^{th} order Butterworth low pass filter. In one example, n is 3.

In some embodiments, the model comprises a classification model for classifying cognitive health state of the subject.

In some embodiments, the model comprises a machine learning based model for determining cognitive health state of the subject. The machine learning based model is trained based on fNIRS data obtained from a population of subjects, which may or may not include the subject. The machine learning based model may be based at least in part on: deep neural network, convolutional neural network, support vector machines, logistic regression, decision trees/forests, ensemble methods (combining models), polynomial/Bayesian/other regressions, stochastic gradient descent, linear discriminant analysis, quadratic discriminant analysis, nearest neighbours classifications/regression, or naïve Bayes, for example.

In some embodiments, determining the cognitive health state of the subject comprises: determining a level of risk of the subject in developing dementia.

In some embodiments, determining the cognitive health state of the subject comprises: determining a stage of dementia the subject is in.

In some embodiments, determining the cognitive health state of the subject comprises: determining whether the subject has subjective memory complaint (SMC). In some embodiments, determining the cognitive health state of the subject comprises: determining a level of severity of the subjective memory complaint (SMC). For example, levels of severity of the SMC may include mild SMC, moderate SMC, and severe SMC.

In some embodiments, determining the cognitive health state of the subject comprises: determining whether the subject has mild cognitive impairment (MCI).

In some embodiments, determining the cognitive health state of the subject comprises: determining whether the subject has amnestic mild cognitive impairment (aMCI).

In some embodiments, determining the cognitive health state of the subject comprises: determining whether the subject has non-amnestic mild cognitive impairment (naMCI).

In some embodiments, the subject is a human subject. In some embodiments, the subject is an animal subject.

In some embodiments, the computer-implemented method further comprises displaying the determination result.

In a second aspect, there is provided a system comprising a processor and memory storing a computer program configured to be executed by the processor. The computer program comprises instructions for performing or facilitating performing of the computer-implemented method of the first aspect. Optionally, the system further comprises a display for displaying one or more of: the fNIRS data, the cognitive task related cerebral hemodynamics data, the determination result (i.e., the determined cognitive health state of the subject), etc.

In a third aspect, there is provided a carrier medium carrying computer readable instructions arranged to cause a computer to perform or facilitate performing of the computer-implemented method of the first aspect. In one example, the carrier medium comprises a computer-readable medium. In one example, the computer-readable medium is a non-transitory computer-readable storage medium, which stores a computer program configured to be executed by a computer. The computer program comprises instructions for performing or facilitating performing of the computer-implemented method of the first aspect.

In a fourth aspect, there is provided a computer program comprising instructions which, when the program is executed by a computer, cause the computer to carry out the computer-implemented method of the first aspect.

In a fifth aspect, there is provided a method comprising: acquiring, using a system operable to perform near-infrared spectroscopy (fNIRS), fNIRS data from a subject when the subject is performing a cognitive task such as a visual memory span task. The fNIRS data contains information associated with cognitive task related cerebral hemodynamics of the subject. The method further comprises: performing the computer-implemented method of the first aspect using the acquired fNIRS data as the fNIRS data.

In a sixth aspect, there is provided a system comprising a system operable to perform near-infrared spectroscopy (fNIRS) and the system of the second aspect. The system operable to perform fNIRS and the system of the second aspect may be operably connected. The system operable to perform fNIRS and the system of the second aspect may be integrated as an integrated system.

Other features and aspects will become apparent by consideration of the following detailed description and the accompanying drawings. Any feature(s) described herein in relation to one aspect or embodiment may be combined with any other feature(s) described herein in relation to any other aspect or embodiment, as appropriate and applicable.

As used herein, unless otherwise specified, terms of degree such that “generally”, “about”, “substantially”, or the like, are intended to account for manufacture tolerance, degradation, trend, tendency, imperfect practical condition(s), etc. Also, unless otherwise specified, the terms “connected”, “coupled”, “mounted” or the like used herein are intended to encompass both direct and indirect connection, coupling, mounting, etc.

### BRIEF DESCRIPTION OF THE DRAWINGS

Some embodiments of the invention will now be described, with reference to the accompanying drawings, in which:

FIG. 1 is a flowchart illustrating a method for determining cognitive health state of a subject in one embodiment of the invention;

FIG. 2 is a flowchart illustrating a method for processing fNIRS data in one embodiment of the invention;

FIG. 3 is a schematic diagram illustrating an operation for determining a cognitive health state of a subject using a model in one embodiment of the invention;

FIG. 4A is a graph showing a receiver operating characteristic (ROC) curve of a data point for detecting individuals with amnestic MCI (compared with normal cognition (NC)) in one embodiment of the invention;

FIG. 4B is a graph showing a ROC curve of a data point for detecting individuals with severe SMC (compared with normal cognition) in one embodiment of the invention;

FIG. 5 is a schematic diagram illustrating a visual memory span task in one embodiment of the invention;

FIG. 6 is schematic diagram illustrating positions of the fNIRS optodes and measurement channels in one embodiment of the invention; and

FIG. 7 is a block diagram of a data processing system operable to perform one or more of the method embodiments in one embodiment of the invention.

### DETAILED DESCRIPTION

Inventors of the present invention have discovered, through their own research and experiments, that cerebral hemodynamics of HbO and HbR can be quite different for individuals with normal cognition (NC), individuals with SMC, individuals with MCI, and individuals with dementia. For example, individuals with MCI and dementia may have decreased cognitive task related HbO changes in the prefrontal brain region when compared with individuals with normal cognition. For example, individuals with SMC may have lower HbO levels during demanding cognitive tasks, hence may have poorer performance on the cognitive tasks. For example, the severity of reduced frontal activation is generally more pronounced in dementia than in MCI, and generally more pronounced in MCI than in SMC. Inventors of the present invention have also devised, through their own research and experiments, that fNIRS, as a non-invasive technique that can be used for measuring cerebral hemodynamics of HbO and HbR, can be useful for determining a cognitive health state of a subject.

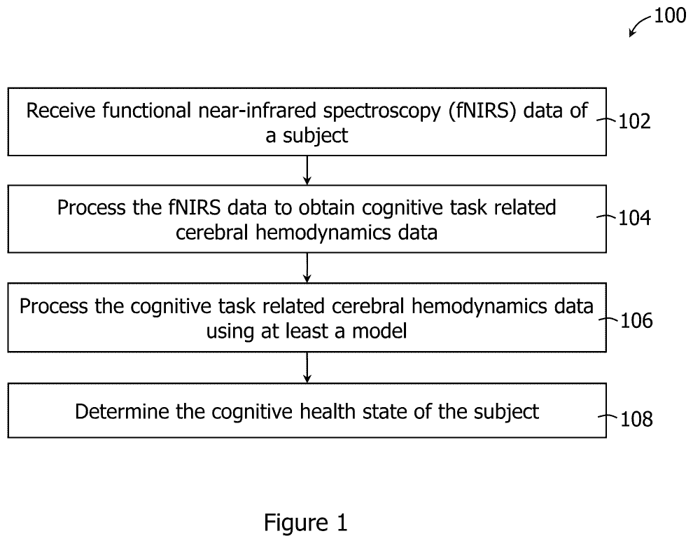

FIG. 1 shows a method 100 for determining cognitive health state of a subject in one embodiment of the invention. The method 100 is a computer-implemented method. In this example, the subject is a human subject.

Method 100 includes operation 102, in which fNIRS data of the subject is received, e.g., at a data processing system. The fNIRS data is acquired from the subject using a system operable to perform fNIRS, when the subject is performing a cognitive task such as a visual memory span task. The fNIRS data contains information associated with cognitive task related cerebral hemodynamics of the subject.

Method 100 further includes operation 104, in which the fNIRS data is processed to obtain cognitive task related cerebral hemodynamics data. Operation 104 can be considered as a pre-processing operation for the fNIRS data. The cognitive task related cerebral hemodynamics data may be associated with hemodynamics of a brain region, including the prefrontal cortex region, of the subject. The cognitive task related cerebral hemodynamics data may include cognitive task related HbO data and/or cognitive task related HbR data. The cognitive task may include a visual memory span task. The visual memory span task may include multiple trials each including a control task and a visual memory span cognitive task.

Method 100 further includes operation 106, in which the cognitive task related cerebral hemodynamics data is processed using at least a model. For example, the model includes a classification model for classifying cognitive health state of the subject. For example, the model includes a machine learning based model for determining cognitive health state of the subject. The machine learning based model may be trained based on fNIRS data obtained from a population of subjects, which may or may not include the subject. The machine learning based model may be based at least in part on: deep neural network, convolutional neural network, support vector machines, logistic regression, decision trees/forests, ensemble methods (combining models), polynomial/Bayesian/other regressions, stochastic gradient descent, linear discriminant analysis, quadratic discriminant analysis, nearest neighbours classifications/regression, naïve Bayes, etc., or any of their combination.

Method 100 further includes operation 108, in which the cognitive health state of the subject is determined based at least in part on the operation 106. For example, operation 108 may include determining a level of risk of the subject in developing dementia. For example, operation 108 may include determining a stage of dementia the subject is in. For example, operation 108 may include whether the subject has SMC. For example, operation 108 may further include determining a level of severity of the SMC. Example levels of severity of the SMC may include mild SMC, moderate SMC, and severe SMC. For example, operation 108 may include determining whether the subject has MCI. For example, operation 108 may include determining whether the subject has aMCI. For example, operation 108 may include determining whether the subject has naMCI.

Method 100 may further include displaying the fNIRS data, the cognitive task related cerebral hemodynamics data, and/or the determination result.

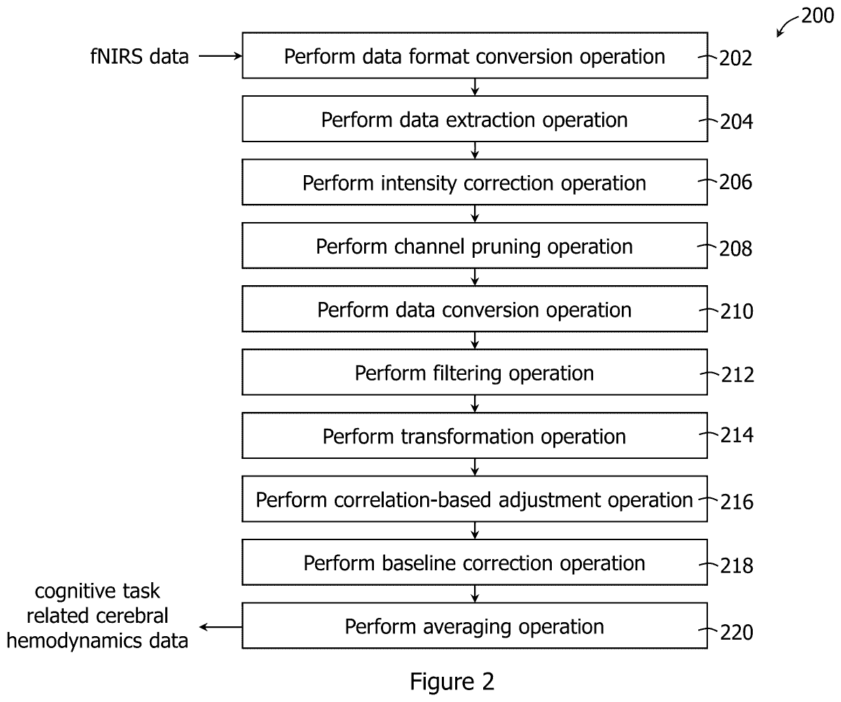

FIG. 2 shows a method 200 for processing fNIRS data in one embodiment of the invention. The method 200 is a computer-implemented method. Method 200 may be considered as an example of operation 104.

Method 200 included operation 202, which includes performing a data format conversion operation. The data format conversion operation converts the fNIRS data from a first data format to a second data format different from the first data format. The second data format may be more suitable (e.g., faster, more secure, etc.) for subsequent processing than the first data format. In one example, operation 202 is not performed if it is determined that no data format conversion is required or that data format conversion is unsuitable.

Method 200 includes operation 204, performed after operation 202 (if operation 202 is performed), which includes performing a data extraction operation on the fNIRS data to obtain, at least, light intensity data, in particular raw light intensity data. In one example, the data extraction operation may extract, from the fNIRS data: parameters associated with source-detector geometry of the system, information associated with stimulus onsets of the cognitive task, information associated source-detector channels of the system, the (raw) light intensity data includes data time points and raw light intensity measurements of each of the channels of the system, and auxiliary signal.

Method 200 includes operation 206, which includes performing an intensity correction operation to remove all negative light intensity values from the light intensity data (obtained from operation 204). In one example, the intensity correction operation includes replacing each negative light intensity value with a respective distance value from 1.0 to a next integer double-precision number. In one example, the intensity correction operation includes, for each negative light intensity value, applying one or more increment signals, e.g., one or more dc signals, to all negative light intensity values in the light intensity data. In one example, operation 206 is not performed if it is determined that there are no negative light intensity values in the light intensity data.

Method 200 includes operation 208, which includes performing a channel pruning operation to prune one or more of the channels (e.g., disregard or remove the data associated with the one or more channels) based at least in part on one or more criteria. In one example, for each channel, the one or more criteria are associated with light intensity values obtained from the channel and one or more thresholds. In one example, for each channel, the one or more criteria are associated with: mean light intensity value of the light intensity values obtained from the channel, standard deviation of light intensity values obtained from the channel, one or more light intensity value thresholds, and a signal-to-noise ratio threshold. In one example, for each channel, the one or more criteria includes at least one of: Mean_{d}>Rangeᵤₚₚₑᵣ, Mean_{d}<Range_{lower}, and

$$\frac{{Mean}_{d}}{S {D}_{d}} < S N {R}_{thresh} ,$$

where Mean_{d} represents mean light intensity value associated with the channel, SD_{d} represents standard deviation of light intensity values associated with the channel, Rangeᵤₚₚₑᵣ represents an upper light intensity value threshold, Range_{lower} represents a lower light intensity value threshold, and SNRₜₕᵣₑₛₕ represents the signal-to-noise ratio threshold, and the channel is pruned if any of the one or more criteria is met. In one example, operation 208 is not performed if it is determined that no channel pruning is required (e.g., as determined based at least in part on the one or more criteria).

Method 200 includes operation 210, which includes performing a data conversion operation on the light intensity data to obtain optical density data. In one example, the data conversion operation is performed based at least in part on:

$$O D = - log \frac{\vert d \vert}{\vert {mean}_{d} \vert}$$

where OD is associated with the optical density data of a channel, d is associated with the light intensity data of the channel, and mean_{d} is associated with mean light intensity of the channel.

Method 200 includes operation 212, which includes performing a filtering operation to at least partly remove noise, in particular high-frequency noise, from the optical density data. The filtering operation may be performed using a filter, such as a bandpass filter or a low pass filter. In one example, the filter includes an n^{th} (e.g., 3^{rd}) order Butterworth low pass filter. In one example, operation 212 is not performed, e.g., if it is determined that the noise is at an acceptable level.

Method 200 includes operation 214, which includes performing a transformation operation on the optical density data to obtain HbO data containing information associated with relative HbO concentration changes and/or HbR data containing information associated with relative HbR concentration changes. In one example, performing the transformation operation includes processing the optical density data based at least in part on modified Beer-Lambert law. In one example, performing the transformation operation includes processing the optical density data based at least in part on absorption coefficient values and distance factors.

Method 200 includes operation 216, which includes performing a correlation-based adjustment operation. In one example, the correlation-based adjustment operation is performed on the HbO data and/or the HbR data to obtain correlation-adjusted HbO data and/or correlation-adjusted HbR data. In one example, the correlation-based adjustment operation includes processing the HbO data and/or the HbR data based at least in part on a correlation function arranged to effectuate a negative correlation between concentration changes of HbO and concentration changes of HbR. In one example, operation 216 is not performed.

Method 200 includes operation 218, which includes performing a baseline correction operation. In one example, the baseline correction operation is performed on the HbO data and/or the HbR data to obtain baseline-corrected HbO data and/or baseline-corrected HbR data. In one example, the baseline correction operation is performed on the correlation-adjusted HbO data and/or the correlation-adjusted HbR data to obtain modified HbO data and/or modified HbR data. In one example, the baseline correction operation includes:

processing the HbO data and/or the HbR data for each trial based at least in part on data obtained during the control task. In one example, operation 218 may be performed prior to operation 216. In one example, operation 218 is not performed.

Method 200 includes operation 220, which includes performing an averaging operation to obtain the cognitive task related cerebral hemodynamics data. The averaging operation may include: averaging the modified HbO data and/or the modified HbR data for each trial based at least in part on data obtained during the visual memory span cognitive task to obtain averaged HbO data and/or averaged HbR data for each trial. The averaging operation may further include: averaging the averaged HbO data and/or averaged HbR data for each trial across trials with the same condition, to obtain cognitive task based HbO data and/or cognitive task based HbR data. The averaging operation may further include: averaging the cognitive task based HbO data and/or cognitive task based HbR data to across all channels that have not been pruned, to obtain the cognitive task related cerebral hemodynamics data.

Various modifications can be made to the method 200 to provide other embodiments of the invention. For example, in some embodiments, one or more of the operations 202-220 can be omitted (i.e., the method may lack one or more of those operations). For example, in some embodiments, the order of the operations 202-220 is different than that illustrated in method 200. That is, in some embodiments, the operations 202-220 can be performed in a different order, as feasible, appropriate, and applicable.

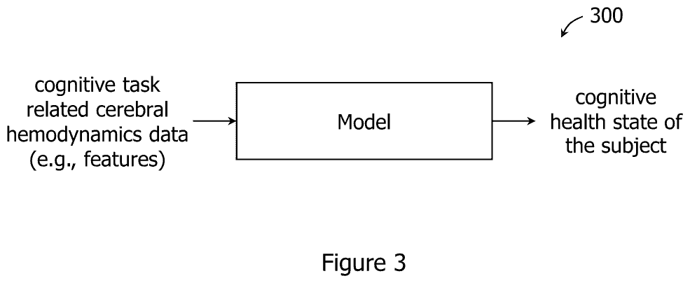

FIG. 3 shows an operation 300 for determining a cognitive health state of a subject using a model in one embodiment of the invention. The operation 300 includes applying the cognitive task related cerebral hemodynamics data (e.g., the cognitive task related cerebral hemodynamics features) into a model to obtain a cognitive health state of the subject. Operation 300 maybe considered as an example of operation 106.

The following provides an embodiment of a method for determining cognitive health state of a subject. The method can be considered as a specific example/implementation of method 100, method 200, and/or operation 300.

In this embodiment, there is provided a functional near-infrared spectroscopy (fNIRS) data processing method for detecting early stages of dementia. The method is a computer-implemented method. The method utilizes fNIRS data collected by a fNIRS system when an individual is performing a computerized visual working memory task paradigm. The objective of this embodiments is to determine whether the individual has early signs of dementia, in particular whether the subject has aMCI, SMC, or normal cognition (NC).

In this embodiment, the fNIRS data processing method generally includes (1) data extraction, (2) replacement of negative raw intensity values, (3) channel pruning, (4) conversion of raw intensity signal to optical density changes, (5) filtering, (6) transformation of concentration of HbO and HbR, (7) correlation-based signal improvement, (8) baseline correction, (9) block averaging, and (10) averaging across time points, conditions, and channels. In this embodiment, the fNIRS data processing method further includes determining whether the individual has early signs of dementia based on cut-off scores. In this embodiment, the cut-off scores are derived based on the study disclosed in Lee et al.'s “fNIRS as a biomarker for individuals with subjective memory complaints and MCI” Alzheimer's Dement. 2024; 1-13, the entire contents of which are hereby incorporated by reference. The cut-off scores serve as reference values for distinguishing individuals with early signs of dementia, i.e., aMCI and severe SMC, from individuals with normal cognition.

The fNIRS data processing method of this embodiment will now be explained in more detail.

First, the fNIRS data processing method includes receiving the fNIRS raw intensity data. In one example, this operation may be an example of operation 102 in method 100.

Then, the format of the fNIRS raw intensity data is converted into the HomER3 data format for subsequent pre-processing, and the following parameters are extracted:
t—data time points array
d—raw intensity time course (measurements).
SD—structured variable containing source/detector geometry, with the following fields:
   Lambda—wavelengths used for data acquisition
   SrcPos—array of probe coordinates of the light sources
   DetPos—array of probe coordinates of the detectors
   nSrcs—number of light sources
   nDets—number of detectors
   MeasList—list of source/detector/wavelength measurement channels
s—time points and condition of stimulus onsets
ml—list of source-detector channels
aux—auxiliary signal of same dimensions as t

In one example, these operations may be an example of operations 202 and 204 in method 200.

Then, the hmrR_PreprocessIntensity_Negative function in MATLAB is employed to address negative intensity values arising from noisy data, as the negative intensity values cannot be further processed. Specifically, for a given d(t):

$$d ( t ) = \{ \begin{matrix}d ( t ) , & d ( t ) > 0 \\ + eps , & d ( t ) \leq 0\end{matrix} \qquad ( 1 )$$

where +eps denotes the distance from 1.0 to the next integer double-precision number. In one example, this operation may be an example of operation 206 in method 200.

Next, the hmrR_PruneChannels function in MATLAB is utilized to prune channels from the measurement list for signals that are either too weak, too strong, or exhibit a significant standard deviation. Specifically, in this embodiment, a channel is pruned when it meets one of the following conditions:

$${Mean}_{d} > {Range}_{upper} \qquad Condition 1$$

$${Mean}_{d} < {Range}_{lower} \qquad Condition 2$$

$$\frac{{Mean}_{d}}{S {D}_{d}} < S N {R}_{thresh} \qquad Condition 3$$

where Rangeᵤₚₚₑᵣ and Range_{lower} represent the upper and lower limits of the range of averaged raw intensity and SNRₜₕᵣₑₛₕ corresponds to the threshold of the signal-to-noise ratio (SNR). In this example, Rangeᵤₚₚₑᵣ and Range_{lower} are set at 100000 and 0 respectively and SNRₜₕᵣₑₛₕ is set at 10. In one example, this operation may be an example of operation 208 in method 200.

Further, the hmrR_Intensity2OD function in MATLAB is used to convert the intensity signal to optical density (OD) changes. Specifically, the conversion is performed based on the following:

$$O D = - log \frac{\vert d \vert}{\vert {mean}_{d} \vert} \qquad ( 2 )$$

In one example, this operation may be an example of operation 210 in method 200.

The hmrR_BandpassFilt function in MATLAB is then used to apply a bandpass filter to the time course data, to remove high-frequency noise. Specifically, in this example, a 3^{rd} order Butterworth low-pass filter with a cutoff frequency of 0.1 Hz is applied. In this example, to perform this function in HomER3, the high pass filter frequency (hpf) and low pass filter frequency (lpf) values are set at 0 and 0.1 respectively. In one example, this operation may be an example of operation 212 in method 200.

The filtered optical density data (OD_{filtered}) is further transformed into changes in relative HbO and HbR concentrations (C) using the modified Beer-Lambert law implemented in the hmrR_OD2Conc function in MATLAB. Specifically, in this example, the following is applied:

$$C = \frac{einv * O {D}_{filtered}}{rho \times ppf} \qquad ( 3 )$$

$$where einv = {( {e}^{T} * e )}^{- 1} * {e}^{T} \qquad ( 4 )$$

In the above, e refers to the absorption coefficient values obtained from the dataset disclosed in Wray et al., “Characterization of the near infrared absorption spectra of cytochrome aa3 and haemoglobin for the non-invasive monitoring of cerebral oxygenation,” Biochimica et Biophsica Acta 1988; 933:184-192. In this example, the wavelengths of 840 nm and 770 nm are specifically selected such that

$$e = [ \begin{matrix}2434.271 & 1789.431 \\ 1615.5545 & 3281.775\end{matrix} ] .$$

In the above, Rho represents the distance between the sources and detectors. In this example, Rho is set as 30. In the above, ppf refers to the partial path length factors for each wavelength. In this example, ppf is set at 6 for both wavelengths. The output of C is a matrix with a t×3 structure for each channel of d. The first column of C represents HbO, the second column represents HbR, and the third column represents total hemoglobin (HbT), where HbT=HbO+HbR. In one example, this operation may be an example of operation 214 in method 200.

In this embodiment, to enhance the signal quality and minimize noise, the hmrR_MotionCorrectCbsi function in MATLAB is employed, utilizing a correlation-based signal improvement (CBSI) technique. Subsequently, the CBSI-corrected HbO and HbR data are corrected based on the principle that the concentration changes of HbO and HbR should exhibit a negative correlation. In this example, the correction is implemented based on the following:

$$cbsiHbO = 0.5 \times ( HbO - \frac{S {D}_{HbO} \times HbR}{S {D}_{HbR}} ) \qquad ( 5 )$$

$$cbsiHbR = - \frac{S {D}_{HbR} \times {HbO}_{CBSI}}{S {D}_{HbO}} \qquad ( 6 )$$

In this example, to enable this function in HomER3, a value of 1 is entered to activate it. In one example, this operation may be an example of operation 216 in method 200.

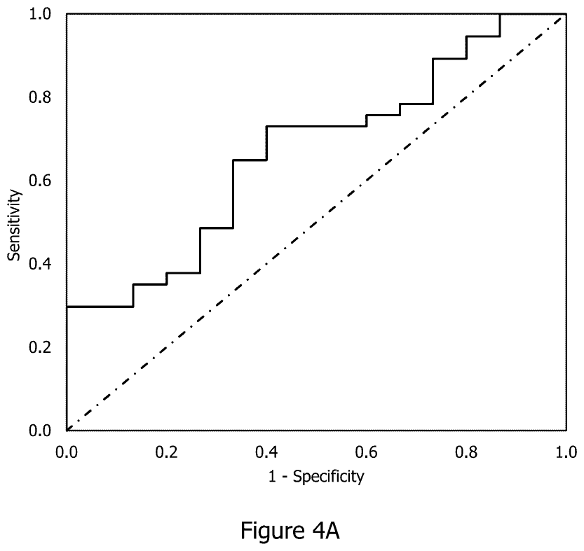

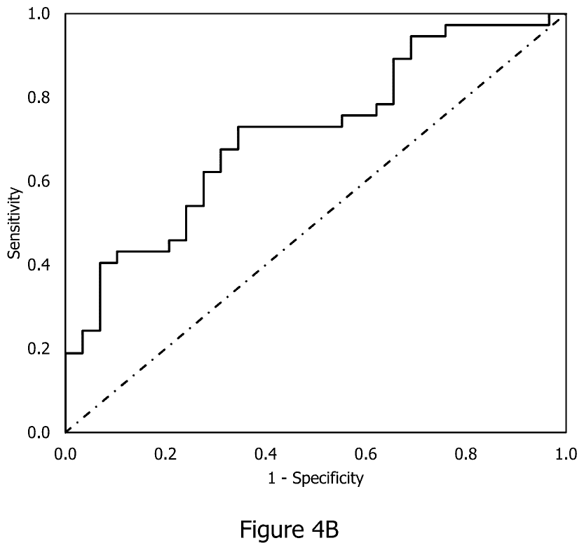

The cbsiHbO and cbsiHbR are then baseline-corrected using the hmrR_BlockAvg function in MATLAB. Specifically, the HbO_{CBSI} and HbR_{CBSI} during the control task period before the start of the visual memory span cognitive task is used for baseline correction. Further details of the visual memory span task containing the control task and the visual memory span cognitive task will be provided with reference to FIG. 4B. In this example, to perform this function, the time range for the block average (t_{range}) is set at [−10 632], with the value of 632 specifically chosen to match the length of a visual working memory span task employed in testing. As a result, the baseline-corrected HbO (bcHbO) and HbR (bcHbR) signals are obtained. In one example, this operation may be an example of operation 218 in method 200.

Next, various averaging operations are performed. In this example, the processed HbO and HbR signals are averaged to obtain the averaged HbO (aHbO) and HbR (aHbR) signals during the visual memory span cognitive task period. Specifically, for a given task period from time t₁ to t₂:

$$aHbO = \frac{{\sum }_{i = {t}_{1}}^{{t}_{2}} {bcHbO}_{i}}{{t}_{{t}_{1} to {t}_{2}}} \qquad ( 7 )$$

$$aHbR = \frac{{\sum }_{i = {t}_{1}}^{{t}_{2}} {bcHbR}_{i}}{{t}_{{t}_{1} to {t}_{2}}} \qquad ( 8 )$$

The denominator (tₜ_{₁}_{to t}_{₂}) refers to the number of time points between time t₁ to t₂, which was calculated by ┌(t₂−t₁)×sampling frequency of fNIRS device┐, with the sampling frequency set at 12.21, as in the OEG-SpO2 system.

In this example, the visual working memory span task is employed, and the values of t₁ and t₂ are specified as follows:

| trial | t₁ (s) | t₂ (s) |
|---|---|---|
| 1 | 10 | 25 |
| 2 | 35 | 50 |
| 3 | 60 | 77 |
| 4 | 87 | 104 |
| 5 | 114 | 138 |
| 6 | 148 | 172 |
| 7 | 182 | 208 |
| 8 | 218 | 244 |
| 9 | 254 | 287 |
| 10 | 297 | 330 |
| 11 | 340 | 375 |
| 12 | 385 | 420 |
| 13 | 430 | 472 |
| 14 | 482 | 524 |
| 15 | 534 | 578 |
| 16 | 588 | 632 |

After obtaining the averaged HbO (aHbO) and HbR (aHbR) signals for each trial, the averaged HbO (aHbO) and HbR (aHbR) signals are further averaged across trials with the same condition to obtain the task-related HbO and HbR signals. In this example, each pair of consecutive trials represents one condition, which corresponds to a specific visual working memory span length. In other words, this example generates task-related HbO and HbR signals for a total of eight conditions, representing span levels 2 to 9, for each channel. In this example, the task-related HbO and HbR signals are further averaged across all available measurement channels. As a result, each individual obtains 16 data points, comprising 8 HbO values and 8 HbR values corresponding to span levels 2 to 9. These data points are denoted as HbOₛₚₐₙ₂ . . . HbOₛₚₐₙ₉ and HbRₛₚₐₙ₂ . . . HbRₛₚₐₙ₉, respectively. In this example, unless certain channels are pruned in the channel pruning operation, the total number of measurement channels remains 16. In one example, these averaging operations may be an example of operation 220 in method 200.

After pre-processing the fNIRS data based on the above operations, the pre-processed fNIRS data is then inputted into a prediction model to determine whether a subject/individual has a severe level of SMC, amnestic MCI, or normal cognition.

In this example, the prediction model utilizes the following classification criteria to determine whether a subject/individual has normal cognition or amnestic MCI:

$$An individual is classified as \{ \begin{matrix}amnestic MCI if {HbO}_{span 9} < 1.12 μM \\ NC if {HbO}_{span 9} \geq 1.12 μM\end{matrix} \qquad ( 9 )$$

In this example, the prediction model utilizes the following classification criteria to determine whether a subject/individual has normal cognition or SMC:

$$An individual is classified as \{ \begin{matrix}severe SMC if {HbO}_{span 8} < 1.25 μM \\ NC if {HbO}_{span 8} \geq 1.25 μM\end{matrix} \qquad ( 10 )$$

The processing using the prediction model and the determination/classification may be an example of operations 106 and 108 in method 100.

In this example, the prediction model is developed based on the findings disclosed in Lee et al.'s “fNIRS as a biomarker for individuals with subjective memory complaints and MCI” Alzheimer's Dement. 2024; 1-13.

FIGS. 4A and 4B show receiver operating characteristic (ROC) curves of the two classifications in this example. Specifically, FIG. 4A shows an ROC curve of HbOₛₚₐₙ₉ for detecting individuals with amnestic MCI from normal cognition whereas FIG. 4B shows an ROC curve of HbOₛₚₐₙ₉ for detecting individuals with severe SMC from normal cognition. For the classification between normal cognition and amnestic MCI, the ROC analysis shows a significant area under the curve (AUC) of 0.67 (p=0.034, FIG. 4A), indicating an accuracy of 69.23%, a sensitivity of 72.97%, and a specificity of 60.00%. For the classification between normal cognition and severe SMC, the ROC analysis shows a significant AUC of 0.71 (p=0.001, FIG. 4B), indicating an accuracy of 69.70%, a sensitivity of 72.97%, and a specificity of 65.52%. The prediction model in this example demonstrates promising results in terms of sensitivity and specificity.

In some embodiments, the prediction model can be implemented as or based on a machine learning based model. For example, to enhance performance of the prediction model, a larger amount of user data can be collected and used for forming or training the model.

For example, by applying machine learning based techniques, the prediction model can be further developed to improve detection accuracy and reliability.

In one example, feature extraction may be utilized. In addition to employing the averaged HbO as a feature for input to the prediction model, other hemodynamic features, such as the standard deviation, slope, skewness, and/or kurtosis of the hemodynamic responses can be extracted and input to the prediction model for classification. These additional features may provide a more comprehensive representation of the hemodynamic features of an individual, potentially capturing more-subtle patterns and improving the overall predictive power of the prediction model.

In one example, N-fold cross-validation may be utilized (e.g., N is an integer larger than 2). In one example, to mitigate the risks of overfitting, a 5-fold cross-validation method can be adopted. The dataset will be randomly partitioned into five groups, with one group serving as the validation set, and the model trained on the remaining four groups. This approach may improve the generalization of the model to unseen data and may avoid over-optimistic performance estimates.

In one example, machine learning model selection may be utilized. For example, the prediction model can be implemented based at least in part on any classification algorithm such as but not limited to decision trees, discriminant analysis, logistic regression classifiers, naïve Bayes classifiers, support vector machines, or nearest neighbour classifiers. For example, the prediction model can be implemented based on two or more of these classification algorithms. In one example, the MATLAB classification learner application can be used to train the model(s) effectively.

In one example, performance evaluation may be applied. To evaluate the performance of each model, a confusion matrix can be generated to display the sensitivity and specificity values.

Furthermore, ROC curve can be plotted for providing a comprehensive visualization of the model's performance. In one example, by comparing these metrics across different models, the most effective model can be identified and selected.

By incorporating a larger dataset and using machine learning based model(s), the accuracy, sensitivity, and specificity of the embodiments of the invention can be improved. An enhanced prediction model can have a broader scope of applicability and may provide more reliable predictions.

In one embodiment, there is provided an fNIRS data processing method for identifying individuals at risk of dementia. The fNIRS data processing method includes converting fNIRS raw intensity data into the HomER3 data format, extracting parameters including time points, raw intensity time course, source/detector geometry, stimulus onsets, source-detector channels, and auxiliary signal. The method further includes addressing negative intensity values in the fNIRS data by replacing them with a distance value from 1.0 to the next integer double-precision number. The method further includes pruning channels from the measurement list based on mean intensity values, standard deviation, and signal-to-noise ratio thresholds. The method further includes converting the intensity signal to optical density changes using the modified Beer-Lambert law. The method further includes applying a 3rd order Butterworth low-pass filter with a cutoff frequency of 0.1 Hz to the data to remove high-frequency noise. The method further includes transforming the filtered optical density data into relative concentrations of HbO and HbR using absorption coefficient values and distance factors. The method further includes enhancing signal quality and minimizing noise through correlation-based signal improvement. The method further includes baseline-correcting the HbO and HbR signals using a control task block before the start of the task. The method further includes averaging the processed HbO and HbR signals during the cognitive task period, averaging the HbO and HbR signals across trials with the same condition, representing visual working memory span lengths, and averaging the task-related HbO and HbR signals across all available measurement channels for each individual, resulting in 16 data points corresponding to different span levels. The method further includes developing a prediction model based on the averaged task-related HbO and HbR signals across all available measurement channels for each individual, and utilizing the prediction model to differentiate individuals who may be at an increased risk of developing dementia (i.e., MCI and SMC).

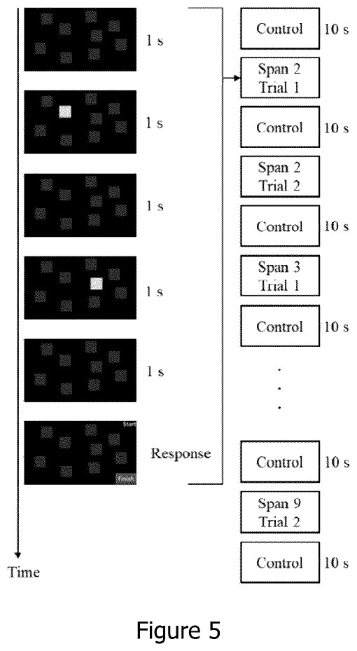

FIG. 5 shows a visual memory span task in one embodiment of the invention. The visual memory span task may be the visual memory span task in method 100 or in the fNIRS data processing method embodiments.

In this example, the visual memory span task used is adapted from a previous fNIRS study disclosed in Lee et al.'s “Prefrontal hemodynamic features of older adults with preserved visuospatial working memory function” Geroscience. 2023; 45 (6): 3513-3527, which investigates the prefrontal hemodynamics of older adults. In this example, as illustrated in FIG. 5, each trial of the visual memory span task begins with a 10-second control task period, during which participants are instructed to focus their attention on a fixation cross displayed at the center of the computer screen. After the control task, nine blue square blocks are presented on the screen for 1 second. Subsequently, the blocks transitions from blue to yellow in a sequential manner, changing color every second. Participants are instructed to memorize the sequence in which the blue blocks are transformed into yellow. After the encoding period, a retrieval phase follows. During this phase, a “start” cue appears in the upper right corner of the screen, accompanied by a “finish” button positioned in the lower right corner. Participants are required to reproduce the sequence by selecting the square blocks on the screen in the same order as they are originally presented. The participants have to click the “finish” button to complete their responses (FIG. 5, left column). The task includes 18 trials, with each span sequence consisting of 2 trials. The span sequences vary in length, ranging from two blocks to nine blocks (FIG. 5, right column). Before the main task, participants undergo two practice trials to familiarize themselves with the task. Stimulus presentation is conducted using PsychoPy version 2022.2.4, as disclosed in Peirce's “PsychoPy-psychophysics software in python”. J Neurosci Methods. 2007; 162 (1-2): 8-13. The visual memory span is calculated based on the longest sequence length that participants accurately reproduced in at least any one trial out of the two trials for each span sequence. It is noted that the task continued even after participants reached their longest correct span length.

The visual memory span task used in this study is based on the Corsi Block-Tapping Test, which originated in the early 1970s as disclosed in Corsi's “Human memory and the medial temporal region of the brain”. Diss Abstr Int. 1972; 34:891, and which has been a common test to assess visual spatial memory in research and clinical settings. Although n-back task is a relatively more common test in neuroimaging studies, in this example, the visual memory span task is selected for two reasons: First, according to the experience of the inventors, some older adults, especially those with low levels of education, have difficulty understanding the complex requirements of n-back tasks. As the visual memory span task instruction is relatively easy for older adults to understand, using the visual memory span task can increase the validity of the results, reducing the possibility that poor performance is due to not understanding the instruction. Second, the visual memory span task generally has nine levels, while the n-back task usually only has three levels. Inventors of the present invention have found that the gradual increase in difficulty level of the visual memory span task makes it a more sensitive measure of hemodynamic change associated with effort.

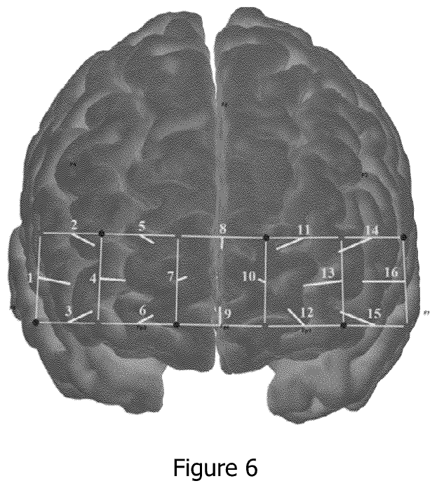

FIG. 6 shows example positions of the fNIRS optodes and measurement channels in one embodiment of the invention. The example positions of the fNIRS optodes and measurement channels may be used to obtain the fNIRS data in method 100, method 200, or the fNIRS data processing method embodiments. In this example, to measure prefrontal hemodynamic activity during the visual memory span task, a 16-channel OEG-SpO2 system (Spectratech Inc.) is used. The system uses near-infrared light with wavelengths of 770 and 840 nm to estimate the relative concentration of HbO in the participants' prefrontal cortex, using the modified Beer-Lambert Law. As shown in FIG. 6, the fNIRS system in this example includes six sources and six detectors arranged in a 2×6 matrix configuration, with a separation distance of 3 cm between the sources and detectors. The center of the bottom probe is positioned approximately on FpZ, following the international 10/20 system. The OEG-SpO2 system has a sampling rate set at 12.21 Hz.

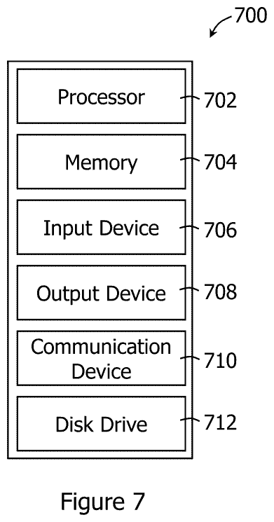

FIG. 7 shows an example data processing system 700 that can be used to perform the method in some embodiments of the invention. For example, the data processing system 700 can be used to perform the method 100 (partly or entirely), or the method 200 (partly or entirely), or the operation 300. The data processing system 700 includes suitable components necessary to receive, store, and execute appropriate computer instructions, commands, and/or codes. In this example, the data processing system 700 includes a processor 702 and a memory 704. The processor 702 may include one or more of: CPU(s), MCU(s), GPU(s), NPU(s), VPU(s), TPU(s), logic circuit(s), Raspberry Pi chip(s), digital signal processor(s) (DSP), application-specific integrated circuit(s) (ASIC), field-programmable gate array(s) (FPGA), and digital and/or analog circuitry (or circuitries) configured to interpret program instructions, to execute program instructions, and/or to process signals and/or information and/or data. The processor 702 may be operable to perform machine learning based processing and non machine learning based processing. The memory 704 may include one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations. Appropriate computer instructions, commands, codes, information and/or data are stored in the memory 704. For example, computer instructions for executing or facilitating executing of the method embodiments of the invention may be stored in the memory 704. For example, the model for assisting in the determination of cognitive health state of the subject in the method embodiments of the invention may be stored in the memory 704. For example, the training/testing/validation data and/or determination result may be stored in the memory 704. The processor 702 and memory 704 may be integrated or separated (and operably connected).

Optionally, the data processing system 700 further includes one or more input devices 706. Examples of an input device 706 include: keyboard, mouse, stylus, image scanner, microphone, tactile/touch input device (e.g., touch sensitive screen), image/video input device (e.g., camera), etc. Optionally, the data processing system 700 further includes one or more output devices 708. Examples of an output device 708 include: display (e.g., monitor, screen, projector, etc.), speaker, headphone, earphone, printer, additive manufacturing machine (e.g., 3D printer), etc. The display may include an LCD display, a LED/OLED display, or other suitable display, which may or may not be touch sensitive. The display may display the data, the processing result, the determination result, etc. The data processing system 700 may further include one or more disk drives 712 which may include one or more of: solid state drive, hard disk drive, optical drive, flash drive, magnetic tape drive, etc. A suitable operating system may be installed in the data processing system 700, e.g., on the disk drive 712 or in the memory 704. The memory 704 and the disk drive 712 may be operated by the processor 702. Optionally, the data processing system 700 also includes a communication device 710 for establishing one or more communication links with one or more other computing devices, such as servers, personal computers, terminals, tablets, phones, watches, IoT devices, or other wireless computing devices. The communication device 710 may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, 6G, or the like) transceiver, an optical port, an infrared port, a USB connection, or other wired or wireless communication interfaces. Transceiver may be implemented by one or more devices (integrated transmitter(s) and receiver(s), separate transmitter(s) and receiver(s), etc.). The communication link(s) may be wired or wireless for communicating commands, instructions, information and/or data. In one example, the processor 702, the memory 704 (optionally the input device(s) 706, the output device(s) 708, the communication device(s) 710 and the disk drive(s) 712, if present) are connected with each other, directly or indirectly, through a bus, a Peripheral Component Interconnect (PCI), such as PCI Express, a Universal Serial Bus (USB), an optical bus, or other like bus structure. In one embodiment, at least some of these components may be connected wirelessly, e.g., through a network, such as the Internet or a cloud computing network. A person skilled in the art appreciates that the data processing system 700 is merely an example and that in other embodiments the data processing system 700 can have a different configuration (e.g., with additional components, fewer components, alternative components, etc.).

Although not required, the embodiments described with reference to the Figures can be implemented as an application programming interface (API) or as a series of libraries for use by a developer or can be included within another software application, operable on such as a terminal or computer operating system or a portable computing device operating system. Generally, as program modules include routines, programs, objects, components and data files assisting in the performance of particular function, the skilled person will understand that the functionality of the software application may be distributed across a number of routines, objects and/or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include any appropriate arrangement of computer or information processing hardware capable of implementing the function described.

Embodiments of the invention have provided various methods and systems for determining cognitive health state of a subject based on functional near-infrared spectroscopy (fNIRS). Embodiments of the invention represent an advancement in the area of dementia detection. Embodiments of the invention provide a valuable, practical tool for early detection in individuals at risk of developing dementia.

Some embodiments of the invention provide a highly sensitive algorithm that utilizes fNIRS, which is a non-invasive, portable, and can be used as a cost-effective biomarker. Some embodiments of the invention have demonstrated the capability of using fNIRS technology and data to accurately distinguish individuals with MCI and SMC, who are at an early risk of developing dementia.

Some embodiments of the invention address the need to identify individuals at risk of dementia, specifically those experiencing MCI and SMC. Some embodiments of the invention provide various pre-processing and/or processing operations, which are particularly useful for differentiating or identifying individuals who may be at an increased risk of developing dementia, enabling early and timely intervention, thereby potentially preventing or ameliorating cognitive decline.

Some embodiments of the invention may be particularly useful for the older population, who are more concerned about cognitive decline and the progression of dementia. Some embodiments of the invention may be particularly suitable for individuals who are proactive in monitoring their cognitive health and seeking early detection of dementia. Some embodiments of the invention may enable accurate and efficient preclinical and early stage detection of dementia, which is particularly useful for related healthcare professionals and medical institutions.

Some embodiments of the invention can be considered as an extension or improvement based on the disclosure in Lee et al.'s “fNIRS as a biomarker for individuals with subjective memory complaints and MCI” Alzheimer's Dement. 2024; 1-13, the entire contents of which are hereby incorporated by reference.

It will be appreciated by a person skilled in the art that variations and/or modifications May be made to the described and/or illustrated embodiments of the invention to provide other embodiments of the invention. The described/or illustrated embodiments of the invention should therefore be considered in all respects as illustrative, not restrictive.

1. A computer-implemented method determining cognitive health state of a subject, comprising:
receiving functional near-infrared spectroscopy (fNIRS) data of a subject, the fNIRS data being acquired using a system operable to perform fNIRS, and the fNIRS data containing information associated with cognitive task related cerebral hemodynamics of the subject;
processing the fNIRS data to obtain cognitive task related cerebral hemodynamics data;
processing the cognitive task related cerebral hemodynamics data using at least a model; and
determining, based at least in part on the processing of the cognitive task related cerebral hemodynamics data, a cognitive health state of the subject.

2. The computer-implemented method of claim 1, wherein the cognitive task related cerebral hemodynamics data is associated with hemodynamics of a brain region, including prefrontal cortex region, of the subject.

3. The computer-implemented method of claim 1, wherein the cognitive task related cerebral hemodynamics data comprises cognitive task related oxyhemoglobin (HbO) data and/or cognitive task related deoxyhemoglobin (HbR) data.

4. The computer-implemented method of claim 1, wherein the processing of the fNIRS data comprises:
(i) performing a data extraction operation on the fNIRS data to obtain light intensity data;
(ii) performing a data conversion operation on the light intensity data to obtain optical density data;
(iii) performing a transformation operation on the optical density data to obtain HbO data containing information associated with relative HbO concentration changes and/or HbR data containing information associated with relative HbR concentration changes;
(iv) performing a correlation-based adjustment operation and a baseline correction operation on the HbO data and/or the HbR data, to obtain modified HbO data and/or modified HbR data; and
(v) performing an averaging operation on the modified HbO data and/or the modified HbR data to obtain the cognitive task related cerebral hemodynamics data.

5. The computer-implemented method of claim 4, wherein performing the data extraction operation comprises:
extracting, from the fNIRS data, parameters associated with source-detector geometry of the system, information associated with stimulus onsets of the cognitive task, information associated source-detector channels of the system, and the light intensity data comprising data time points and raw light intensity measurements of each of the channels of the system.

6. The computer-implemented method of claim 5, wherein performing the data extraction operation further comprises:
further extracting, from the fNIRS data, auxiliary signal.

7. The computer-implemented method of claim 4, wherein the data conversion operation is performed based at least in part on:
$$O D = - log \frac{\vert d \vert}{\vert {mean}_{d} \vert}$$
where OD is associated with the optical density data of a channel, d is associated with the light intensity data of the channel, and mean_{d} is associated with mean light intensity of the channel.

8. The computer-implemented method of claim 4, wherein performing the transformation operation comprises:
processing the optical density data based at least in part on modified Beer-Lambert law.

9. The computer-implemented method of claim 4, wherein performing the transformation operation comprises:
processing the optical density data based at least in part on absorption coefficient values and distance factors.

10. The computer-implemented method of claim 4, wherein performing the correlation-based adjustment operation and the baseline correction operation comprises:
performing the correlation-based adjustment operation on the HbO data and/or the HbR data to obtain correlation-adjusted HbO data and/or correlation-adjusted HbR data; and
performing the baseline correction operation on the correlation-adjusted HbO data and/or the correlation-adjusted HbR data to obtain the modified HbO data and/or the modified HbR data.

11. The computer-implemented method of claim 4, wherein performing the correlation-based adjustment operation comprises:
processing the HbO data and/or the HbR data based at least in part on a correlation function arranged to effectuate negative correlation between concentration changes of HbO and concentration changes of HbR.

12. The computer-implemented method of claim 4,
wherein the cognitive task comprises a visual memory span task including a plurality of trials, each trial comprising a control task and a visual memory span cognitive task.

13. The computer-implemented method of claim 12, wherein performing the baseline correction operation comprises:
processing the HbO data and/or the HbR data for each trial based at least in part on data obtained during the control task.

14. The computer-implemented method of claim 12, wherein performing the averaging operation comprises:
averaging the modified HbO data and/or the modified HbR data for each trial based at least in part on data obtained during the visual memory span cognitive task to obtain averaged HbO data and/or averaged HbR data for each trial;
averaging the averaged HbO data and/or averaged HbR data for each trial across trials with the same condition, to obtain cognitive task based HbO data and/or cognitive task based HbR data; and
averaging the cognitive task based HbO data and/or cognitive task based HbR data to across all channels that have not been pruned.

15. The computer-implemented method of claim 4, wherein the processing of the fNIRS data further comprises:
prior to (ii), performing an intensity correction operation to remove all negative light intensity values from the light intensity data.

16. The computer-implemented method of claim 15, wherein performing the intensity correction operation comprises:
replacing each negative light intensity value with a respective distance value from 1.0 to a next integer double-precision number.

17. The computer-implemented method of claim 4, wherein the processing of the fNIRS data further comprises:
performing a channel pruning operation to prune one or more of the channels based at least in part on one or more criteria.

18. The computer-implemented method of claim 17, wherein the channel pruning operation is performed prior to (ii).

19. The computer-implemented method of claim 17, wherein for each channel, the one or more criteria are associated with light intensity values obtained from the channel and one or more thresholds.

20. The computer-implemented method of claim 19, wherein for each channel, the one or more criteria are associated with:
mean light intensity value of the light intensity values obtained from the channel;
standard deviation of light intensity values obtained from the channel;
one or more light intensity value thresholds; and
a signal-to-noise ratio threshold.

21. The computer-implemented method of claim 20,
wherein for each channel, the one or more criteria comprises at least one of:
$${Mean}_{d} > {Range}_{upper} ,$$
$${Mean}_{d} < {Range}_{lower} , and$$
$$\frac{{Mean}_{d}}{S {D}_{d}} < S N {R}_{thresh}$$
where Mean_{d} represents mean light intensity value associated with the channel, SD_{d} represents standard deviation of light intensity values associated with the channel, Rangeᵤₚₚₑᵣ represents an upper light intensity value threshold, Range_{lower} represents a lower light intensity value threshold, and SNRₜₕᵣₑₛₕ represents the signal-to-noise ratio threshold, and the channel is pruned if any of the one or more criteria is met.

22. The computer-implemented method of claim 4, wherein the processing of the fNIRS data further comprises:
prior to (iii), performing a filtering operation to at least partly remove noise from the optical density data.

23. The computer-implemented method of claim 22, wherein performing the filtering operation comprises:
processing the optical density data using a bandpass filter or a low pass filter.

24. The computer-implemented method of claim 1, wherein the model comprises a classification model for classifying cognitive health state of the subject.

25. The computer-implemented method of claim 1, wherein the model comprises a machine learning based model for determining cognitive health state of the subject.

26. The computer-implemented method of claim 1, wherein determining the cognitive health state of the subject comprises:
determining a stage of dementia the subject is in.

27. The computer-implemented method of claim 1, wherein determining the cognitive health state of the subject comprises:
determining whether the subject has subjective memory complaint (SMC) and optionally a level of severity of the SMC.

28. The computer-implemented method of claim 1, wherein determining the cognitive health state of the subject comprises:
determining whether the subject has mild cognitive impairment (MCI).

29. The computer-implemented method of claim 1, wherein determining the cognitive health state of the subject comprises:
determining whether the subject has amnestic mild cognitive impairment (aMCI).

30. The computer-implemented method of claim 1, wherein determining the cognitive health state of the subject comprises:
determining whether the subject has non-amnestic mild cognitive impairment (naMCI).
